(19) United States
(12) Patent Application Publication
(10) Pub. No.: US 2009/0054517 A1
(43) Pub. Date: Feb. 26, 2009

(54) Phytoestrogens As Regulators Of Hedgehog Signaling And Methods Of Their Use In Cancer Treatment

(76) Inventors: Dennis B. Lubahn, Columbia, MO (US); Anna Slusarz, Columbia, MO (US); Nader Shenouda, Winnipeg (CA); Mary S. Sakla, Winnipeg (CA)

Correspondence Address: LATHROP & GAGE LC, 4845 PEARL EAST CIRCLE, SUITE 300, BOULDER, CO, 80301, US

(21) Appl. No.: 12/106,993

(22) Filed: Apr. 21, 2008

Related U.S. Application Data
(60) Provisional application No. 60/925,661, filed on Apr. 20, 2007.

(51) Int. Cl. A61K 31/352; C12Q 1/02

(52) U.S. Cl. 514/456; 435/29

(57) ABSTRACT

A new method is provided for inhibiting tumor growth and for delaying the onset of cancer. Several estrogenic compounds from plants are capable of inhibiting cell proliferation both in cell cultures and in whole animals. These compounds likely exert their anti-proliferation effects by inhibiting the Hedgehog signaling pathway. Estrogen receptors may also play an essential role in the inhibitory effect of these compounds.

### RELATED APPLICATION

This application claims priority to U.S. provisional application Ser. No. 60/925,661 filed Apr. 20, 2007, which is hereby incorporated by reference.

### BACKGROUND

1. Field of the Invention

This disclosure pertains to prevention and suppression of tumor growth. More particularly, it relates to the use of phytoestrogens to delay and/or treat certain types of cancer.

2. Description of Related Art

Cancer is one of the top health-related causes of human death. Prostate cancer is the most common form of non-skin cancer among men in the United States. See generally, Jemal et al. 2007. The incidence of prostate cancer is believed to be controlled primarily by endocrine and dietary factors, as well as by genetic factors. Because prostate cancer typically develops later in life, identifying compounds that delay or inhibit the progression of this disease will have a significant impact on the incidence of this malignancy among the aging male population. Furthermore, because prostate cancer typically responds, at least initially, to endocrine and dietary mediators, new compounds that may inhibit tumor growth may be used in chemotherapy for treating patients who have already developed prostate cancer.

Despite extensive search for plant derived compounds (“botanical compounds”) that may delay and/or suppress cancerous growth, only a small number of plant derived candidate cancer cures/treatments have been identified. One of these is cyclopamine, which was originally isolated from Veratrum californicum in the 1960s (Keeler et al., 1970). Cyclopamine has recently been shown to result in complete regression of xenograft tumors in mice (Karhadkar et al., 2004).

Although cyclopamine exists naturally, it has been known to cause birth defects, most notably cyclopia, namely, the fusion of two eyes into one central eye in the progeny (Keeler et al., 1970). Thus, the use of cyclopamine in the laboratory or in a clinical setting may pose a hazard to health care professionals, patients and researches. In addition, cyclopamine is very expensive and may not be as effective as has initially been suggested. Recent reports suggest that the effects of cyclopamine may be limited only to PDC (poorly differentiated carcinoma) type or highly aggressive tumors in mice. See—Sanchez et al. 2005, Karhadkar et al. 2004. There is therefore a need for a safer, less expensive and more effective compound for treating and/or preventing prostate cancer.

Several lines of evidence suggest that soy products may contain such a compound that may serve as a superior alternative for cyclopamine. Dietary consumption of soy products has long been associated with reduced incidence of diseases such as cancer. A number of epidemiological studies have suggested that dietary intake of soy products may play a role in preventing prostate cancer. See Goetzl et al. 2007. The two primary isoflavone aglycones in soy are genistein and daidzein, which are heterocyclic phenols with structural homology to 17β-estradiol. Because they possess estrogenic activity and are derived from plant sources, they are also known as phytoestrogens. Soy foods lacking isoflavones lose almost all cancer protection activity, suggesting that the isoflavones may be the key ingredient in soy that confers the anti-cancer activity (Barnes et al, 1994).

Numerous models have been proposed to illuminate the underlying mechanisms through which soy isoflavones may affect the incidence of cancer or may interact with tumor cells. For instance, genistein has been found to be an inhibitor of tyrosine kinases, which play a critical role in tyrosine phosphorylation, signal transduction, mitogenic regulation, and cell cycle progression. Genistein has also been shown to inhibit angiogenesis. In addition to its inhibition of mitochondrial aldehyde dehydrogenase, which may limit tumor cell growth, genistein acts as a potent antioxidant and inhibits topoisomerase II. Further, genistein induces apoptosis, which may limit tumor cell growth and metastasis.

Last but not least, genistein may also act as an estrogen, interfering with and potentially altering growth and differentiation responses mediated through estrogen receptor (ER), including estrogen receptor alpha (ERα) and/or beta (ERβ). Soy genistein has been shown to bind both receptors with a higher affinity for ERβ and initiate transcription of ER-dependent genes (Kuiper 1997). Dietary soy has been shown to inhibit the effects of neonatal estrogenization of the mouse prostate, perhaps by acting as a weak estrogen and blocking the effects of diethylstilbestrol (DES) through an ERα or ERβ dependent-pathway. In the rat dorsolateral prostate, genistein was shown to inhibit the expression of epidermal growth factor (EGF) and ErbB2/Neu receptors.

Studies in cultured cells have demonstrated that soy phytoestrogens, such as genistein, inhibit the growth of prostate cancer cell lines in vitro. See Shenouda et al. 2004. Previous work by Mentor-Marcel et al (2001) identified a protective effect of genistein in reducing poorly differentiated carcinoma (PDF) in the TRAMP (Transgenic Adenocarcinoma of Mouse Prostate) mouse model.

Together these studies suggest that phytoestrogens such as genistein may play an important role in the prevention or inhibition of cancer development. However, no studies have directly characterized the responses of normal and cancer cells to these phytoestrogens in vivo. More importantly, no studies have directly linked phytoestrogens and their anti-cancer activity to an underlying cellular pathway that is implicated in cell proliferation or cell differentiation (Karhadkar et al., 2004). There is therefore a need for an established method whereby phytoestrogens may be administered to an individual for preventing and/or treating cancer.

### SUMMARY

The instrumentalities described herein advance the art by providing a new composition which may be used to prevent and/or treat cancer. The disclosure further provides a novel method for regulating the Hedgehog signaling pathway.

Recent studies have implicated Hedgehog signaling pathway in a variety of malignancies. The Sonic Hedgehog (Shh) signal transduction pathway (also called the “Hedgehog signaling pathway”) is crucial to the growth, survival and organization of many cells, tissues and organs. For review, see Datta (2006). The deregulation of the Shh signal-transduction pathway has been implicated in several cancers, including prostate cancer, basal cell carcinoma, medulloblastomas, glioma, sarcoma, colorectal cancer, tumors of the digestive tract, small cell lung cancer and pancreatic carcinoma (Sanchez et al., 2005).

Briefly, components of the Hedgehog-signaling pathway, namely, SHH, PTCH, GLI, and in some cases, SMO, have been reported to be elevated in metastatic prostatic tumors versus normal prostates or non-metastatic tumors. Inhibitors of the Hedgehog pathway, for example, cyclopamine or anti-SHH antibodies, have been shown to inhibit the growth of several well known human prostatic cell lines in vitro, including LNCaP and PC-3 (Sanchez et al., 2004). More importantly, in multiple xenograft models these same inhibitors worked extraordinarily well to inhibit tumor growth and metastases in vivo. According to Ruiz I Altaba et al., the Hedgehog pathway culminates in the activation of transcription factors of the Gli family, namely, the Gli1, Gli2 and Gli3 proteins (2002). Therefore, inhibition of Gli function might be a promising therapeutic target in certain tumors.

Little work has been done to explore any potential cross-talk between phytoestrogens and the Hedgehog signaling pathway. The present disclosure shows for the first time that a number of phytoestrogens, including genistein, may inhibit the Hedgehog pathway, which may in turn lead to inhibition of tumor growth. It is shown that LNCaP and PC-3 cell proliferation may be regulated by a number of these phytoestrogens, such as genistein and EGCG. TRAMP-C2 cells, which are derived from a mouse model for prostate cancer, TRAMP, may be used to study the effect of these phytoestrogens on cell proliferation and the underlying mechanisms. The proliferation of TRAMP-C2 cells may be inhibited when they are treated with certain phytoestrogens, including genistein and EGCG.

Most of the components of the Hedgehog signaling pathway are expressed in the TRAMP-C2 cells, suggesting that the pathway is likely to be activated in these cells. A number of phytoestrogens, including, for example, genistein and EGCG may down-regulate the expression of Gli1, Gli2 and Gli3 in the TRAMP-C2 cells.

It has previously been shown that soy genistein, ERα (estrogen receptor α) and ERβ have dramatically different effects on tumorigenesis in the TRAMP mouse model. Additionally, a recent paper suggests that ERβ, but not ERα, works through the dihydrotestosterone metabolite, 5α-androstane-3β,17β-diol, to regulate E-cadherin in prostate cancer (Guerini, 2005). Soy phytoestrogen preferentially binds to ERβ over ERα.

It is hereby disclosed a composition useful for treating cancer, or for preventing the incidence or the recurrence of cancer in an individual. The composition may contain an estrogenic agent in an effective amount that is capable of inhibiting the Hedgehog signaling pathway in a cell.

In another embodiment, a method is provided for treating cancer, or for preventing the incidence or the recurrence of cancer in an individual. The method may comprise the step of administering to said individual an effective amount of an estrogenic agent, wherein said estrogenic agent in said effective amount is capable of inhibiting the Hedgehog signaling pathway in certain cells of said

This disclosure also provides another method for inhibiting Hedgehog signaling pathway in an individual with aberrant Hedgehog signaling. This method may include the steps of (a) determining whether Hedgehog signaling is abnormal in said individual, and (b) administering to said individual a Hedgehog signaling inhibiting effective amount of an estrogenic agent. Alternatively, this disclosure also provides a method for inhibiting Hedgehog signaling pathway in a cell, comprising contacting said cell with an estrogenic agent, said estrogenic agent being present in an amount that is effective in inhibiting expression of at least one Gli gene by at least 40%.

Examples of estrogenic agents may include estrogens or their analogs, derivatives, esters, pharmaceutically acceptable salts, hydrates, or any combination thereof. For purpose of this disclosure, the estrogen may be one that is produced by an animal, a plant, a fungal species, or a human being. The estrogen can be also produced by chemical synthesis or by biological engineering. In a preferred embodiment, the estrogen is a phytoestrogen. In one aspect, the composition may contain one or more members of phytoestrogens selected from the group consisting of Genistein, Quercetin, Baicalein, Apigenin, Daidzein, Neoxanthin, Spinacetin, Patuletin, Luteolin, Curcumin, Resveratrol, EGCG and derivatives thereof.

In one aspect, the effective amount of an estrogenic agent is the amount of said estrogenic agent that can substantially reduce Hedgehog signaling in a cell. In another aspect, the effective amount of said estrogenic agent is the amount that is capable of binding to an estrogen receptor in said individual thereby inhibiting the Hedgehog signaling pathway in said individual. In another aspect, the effective amount of the estrogenic agent may be an amount of the estrogenic agent that is effective in reducing the expression of at least one Gli gene by at least 40%. For purpose of this disclosure, the term “at least one Gli gene” means at least one member selected from the group consisting of Gli1, Gli2 and Gli3.

It is also disclosed a method for treating cancer, or for preventing the incidence or the recurrence of cancer in an individual, comprising administering to said individual an effective amount of an agent, wherein said agent in said effective amount is capable of binding at least one estrogen receptor in the individual, thereby inhibiting the Hedgehog signaling pathway in said individual. For purpose of this disclosure, the agent may be an estrogenic agent, or any chemical compounds or proteins or polynucleotides that can bind an estrogen receptor to trigger downstream signaling events that include but are not limited to the Hedgehog signaling pathway.

In yet another embodiment, the present disclosure provides a screening method useful for identifying potential therapeutic agents that may be useful in treating or preventing cancer. The method may include the steps of: (a) contacting a cell with an intact Hedgehog signaling pathway with a candidate molecule; (b) monitoring changes in the activation of the Hedgehog signaling pathway; (c) determining whether said molecule is capable of inhibiting the Hedgehog signaling pathway. (d) identifying said molecule as a potential therapeutic agent if it is determined to be capable of inhibiting the Hedgehog signaling pathway. Compounds identified according to this screening method may be used for treating cancer, or for preventing the incidence or the recurrence of cancer in an individual.

### BRIEF DESCRIPTION OF THE FIGURES

FIG. 1 illustrate the chemical structures of some representative phytoestrogens along with the structures of 17β-estradiol and cyclopamine.

FIG. 2 shows expression of components of the Hedgehog-signaling pathway mRNA by RT-PCR in TRAMP-C2 cells.

FIG. 3 shows expression of estrogen receptors mRNA by RT-PCR in the TRAMP-C2 cells.

FIG. 4 shows the effects of seven phytoestrogens on the growth of TRAMP C2 cell line by measurement of total cellular protein.

FIG. 5 shows inhibition of Gli1 mRNA expression by the phytoestrogens by RT-PCR.

FIG. 6 shows results of a dose response study on Gli1 mRNA by real-time RT-PCR in which TRAMP C2 cells are treated with increasing amounts of genistein or EGCG.

FIG. 7A shows the result of a time course experiment in which the changes in Gli1 levels in TRAMP-C2 cell line were followed for 72 hrs in the absence or presence of various phytoestrogens. FIG. 7B shows the effects of various phytoestrogens on Hedgehog signaling in a non-tumor cell line Shh Light II. FIG. 7C shows that Estradiol (E2) inhibits induction of Gli1 transcription after 24 hours in TRAMP-C2 cells.

FIG. 8 shows immunohistochemistry of TRAMP prostate tissues.

FIG. 9 shows PDC “Neuroendocrine-like” Neoplasia Progression in Prostate Cancer.

### DETAILED DESCRIPTION

It is to be understood that this disclosure teaches by way of example, and not by limitation. The instrumentalities described herein are broader than the particular methods and materials, which may vary within the skill of the art. It is also to be understood that the terminology used herein is for the purpose of describing particular embodiments only, and is not intended to be limiting. Names of chemicals, proteins, genes and pathways may be referred to using either small or capital letters, which shall be interpreted to refer to the same. As used throughout this disclosure, references by author name and publication year are more particularly cited in the References section. Further, unless defined otherwise, all technical and scientific terms used herein have the same meaning as commonly understood by one of ordinary skill in the related art. The following terminology and grammatical variants are used in accordance with the definitions set out below.

The materials and methods described herein relate to the use of phytoestrogens to prevent or to treat certain cancers. Although prostate cancer is used as a model for the present disclosure, the compositions and their therapeutic use may be applicable to other cancer types, including but not limited to breast cancer, colon cancer, and testicular cancer. The compositions may contain, in addition to the disclosed phytoestrogens, other components which are compatible with the phytoestrogen(s) to be given and may enhance the delivery, absorption, clinical effects or other aspects of the actions by such phytoestrogens. For instance, one may find it desirable to modify the phytoestrogens by conjugation or other methods to make them more stable. The compositions may be administered to a subject as medicinal pills, liquid injectibles or as dietary supplements. The methods of delivery may include, for example, oral delivery, blood injection, local injection or inhalation.

In another aspect, this disclosure relates to the treatment and prevention of cancer. More particularly, the present disclosure provides 1) methods of preventing carcinogenesis in an individual; 2) methods of preventing the recurrence of, suppressing, inhibiting or reducing the incidence of carcinogenesis in an individual; 3) methods of treating an individual with cancer; 4) methods of suppressing, inhibiting or reducing the incidence of cancer in an individual; 5) methods of treating an individual with pre-malignant lesions of cancer; and 6) methods of suppressing, inhibiting or reducing the incidence of pre-malignant lesions of cancer in an individual, by administering to the individual a phytoestrogen, and/or an analog or metabolite thereof, derivative, ester, pharmaceutically acceptable salt, hydrate, or any combination thereof.

In one embodiment, the present disclosure provides a safe and effective method for delaying or preventing the onset of prostate cancer, and is useful for treating an individual having an elevated risk of developing prostate cancer, for example, those having benign prostatic hyperplasia, prostate intraepithelial neoplasia (PIN), or an abnormally high level of circulating prostate specific antigen (PSA), or having a family history of prostate cancer.

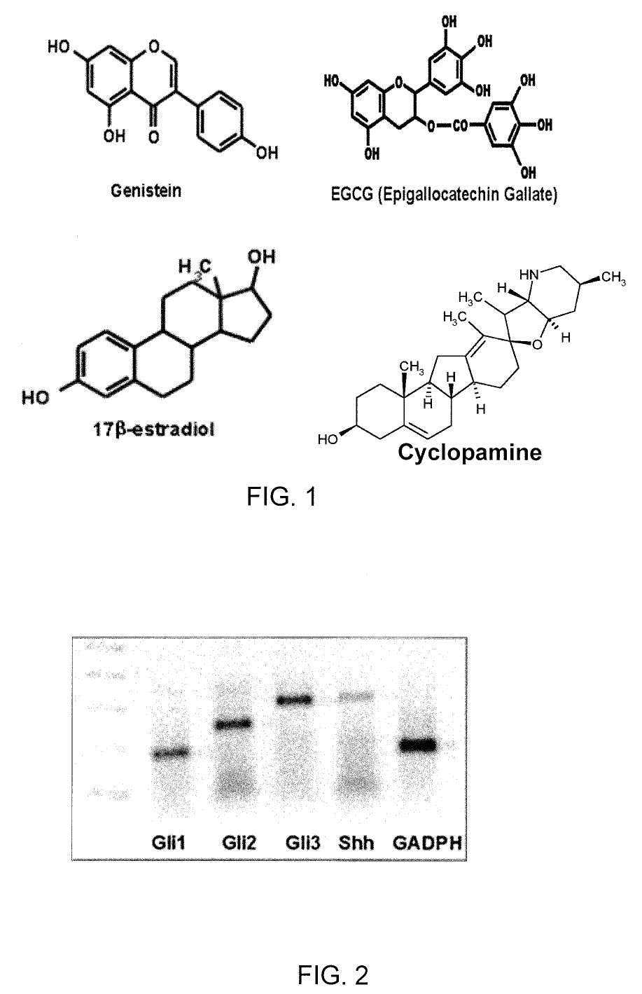

This disclosure provides a method for administering to a subject an effective dose of a phytoestrogen to treat, prevent, suppress recurrence of, and/or inhibit prostate cancer and to treat, prevent, suppress recurrence of, and/or inhibit pre-malignant lesions of prostate cancer. In one embodiment, the phytoestrogen is a compound belonging to the class of heterocyclic phenols, which are structurally similar to 17β-estradiol. Suitable compounds may include, for examples, Genistein, Quercetin, Baicalein, Apigenin, Daidzein, Neoxanthin, Spinacetin, Patuletin, Luteolin, Curcumin, Resveratrol, EGCG and derivatives thereof. Of these, Genistein, Quercetin, Baicalein, Spinacetin, Patuletin, Luteolin and Apigenin may be categorized as isoflavones. Chemical structures of some representative phytoestrogens are shown in FIG. 1 along with the structures of 17β-estradiol and cyclopamine.

The term “estrogenic agent” refers to a chemical, a biologic, an organism, a mixture, an extract or combination thereof that may act as an estrogen or such an agent that may possess or may be induced to demonstrate a detectable estrogenic activity under certain conditions. The term “estrogenic activity” refers to a biological activity that is typically demonstrated by an estrogen when administered to an individual, regardless of whether the estrogen is produced by the individual or by other sources, such as plants or cell cultures. A phytoestrogen refers to an estrogenic agent that may be derived or prepared from any parts of a plant.

It is to be understood that the estrogenic agents of the present disclosure may be derived from natural resources such as plants, fungi or other organisms. In a preferred embodiment, the estrogenic agent is a phytoestrogen. Alternatively, the estrogenic agents of the present disclosure may be prepared through chemical synthesis or biological engineering. For purpose of this disclosure, biological engineering may refer to activities including manipulation of a living cell in order to alter the behavior of the cell or to induce the cell to produce certain molecules in a quantity that is not typically produced by such a cell.

In another embodiment, the methods of the present disclosure comprise administration to a subject a pharmaceutically acceptable salt, ester, N-oxide, hydrate or mixtures thereof of the disclosed phytoestrogens and analogs thereof. In another embodiment, the methods of the present disclosure comprise administration to a subject an analog and/or metabolite of the disclosed phytoestrogens and analogs thereof. A composition and/or a pharmaceutical composition may also be prepared containing the disclosed phytoestrogens and analogs thereof.

Thus, the present disclosure provides a method of preventing carcinogenesis in a subject by oral administration to the subject a pharmaceutical composition containing from about 100 mg to about 600 mg of a phytoestrogen per kilogram (kg) by weight of the subject, its ester or other derivatives, pharmaceutically acceptable salt, hydrate, or any combination thereof and/or an analog or metabolite thereof.

In another embodiment, this disclosure provides a method of preventing the recurrence of, suppressing, inhibiting or reducing the incidence of prostate carcinogenesis, or increasing the survival rate of a subject having prostate cancer, said method including the step of oral administering to said subject a pharmaceutical composition containing from about 100 mg to about 600 mg of a phytoestrogen per kg by weight of the subject, its ester or other derivatives, pharmaceutically acceptable salt, hydrate, or any combination thereof and/or an analog or metabolite thereof.

In another embodiment, this disclosure provides a method of treating cancer, preventing the recurrence of, suppressing, inhibiting or reducing the incidence of prostate carcinogenesis, or increasing the survival rate of a subject having cancer, said method including the step of oral administering to said subject a dietary composition containing from about 10 mg to about 10 grams of a phytoestrogen, its ester or other derivatives, pharmaceutically acceptable salt, hydrate, or any combination thereof and/or an analog or metabolite thereof, per kg of diet to be consumed by said subject. It is to be understood that the amount of said phytoestrogen in the composition may be increased if a more drastic effect is desired up to a maximum dose that can be tolerated by the subject.

In yet another embodiment, the pharmaceutical composition containing a phytoestrogen may be administered by injection or other methods in such an amount so that the phytoestrogen may be delivered to a site within the subject's body and form a local concentration at the site that is sufficient to suppress the Gli1 gene expression by at least 40%. By way of example, the concentration of phytoestrogen in cells at such a body site may be in the range of about 20-100 μM in the case of genistein, and about 10-150 μM in the case of EGCG. These ranges may be considered an effective concentration of the respective phytoestrogen in a cell that may effectively inhibit the Hedgehog signaling pathway in such a cell. For purpose of the present disclosure, a cell proliferation inhibiting effective amount refers to the amount of an agent that, when applied to a cell culture or when administered to an individual, is capable of reducing the rate of cell growth by at least 30%.

U.S. Patent application publication No. 20050090457 by Schoenmakers et al. teaches a method for treating mammals with genistein and/or genistein analogues. A report by Takimoto et al. (2003) describes the pharmacokinetic and pharmacodynamic of unconjugated soy isoflavones administered to individuals with cancer. These references are hereby expressly incorporated by reference.

In one aspect of this disclosure, the subject may have an elevated risk of prostate cancer. In another aspect, the subject may have benign prostatic hyperplasia, prostatic intraepithelial neoplasia (PIN), or an abnormally high level of circulating prostate specific antigen (PSA). A pre-administration screening may also be conducted wherein each individual is subject to a test to determine if the Hedgehog signaling is sufficiently abnormal to justify treatment with the phytoestrogens of the present disclosure. Such screening may be performed by, for example, testing the mRNA or protein levels of various proteins in the Hedgehog pathway. The term “abnormal” means the level of a molecule of interest deviates from that in a normal cell by at least 20%. For example, if the Gli1 mRNA levels are shown to be more than 20% higher than normal cells from the same individual or from comparable individuals of the general population, such individual may be characterized as having abnormal Hedgehog signaling, at least with respect to the tissue or organ tested. The genes encoding the Gli family proteins may be collectively referred to as the Gli genes.

For purpose of this disclosure, the terms “individual” and “subject” are used interchangeably, and may refer to a mammal, such as human or other animals.

The TRAMP mice used for the animal studies are as described by Greenberg et al., 1995. The TRAMP C2 cell lines derived from the TRAMP mice are as described by Foster et al., 1997. Various plant derived agents may be prepared directly from the respective plant sources. To maintain a consistent standard with respect to the compounds used, for purpose of this disclosure, the compounds are obtained from commercial sources. Cyclopamine is obtained from LC laboratories (it can also be obtained from Toronto chemicals), while genistein and EGCG are both from Sigma Co.

In one aspect of the present disclosure, in order to determine the effect of genistein on prostate tumor development in the TRAMP mice and the underlying mechanisms, if any, ERαKO/TRAMP (KO=knock-out), ERβKO/TRAMP or ERWT/TRAMP mice are fed diets containing 0 or 300 mg genistein/kg of diet. A significant protective effect of genistein is observed in the ERWT/TRAMP mice but not in the ERαKO/TRAMP and ERβKO/TRAMP mice, suggesting that both ERα and ERβ may be essential for the response to this class of phytoestrogens, including genistein.

### EXAMPLES

The following nonlimiting examples disclose general procedures, reagents and characterization methods that teach by way of example, and should not be construed in a narrowing manner that limits the disclosure to what is specifically disclosed. Those skilled in the art will understand that numerous modifications may be made and still the result will fall within the spirit and scope of the present invention.

### Example 1

### TRAMP-C2 Cell Line Expression of Estrogen Receptors and Hedgehog Path Components

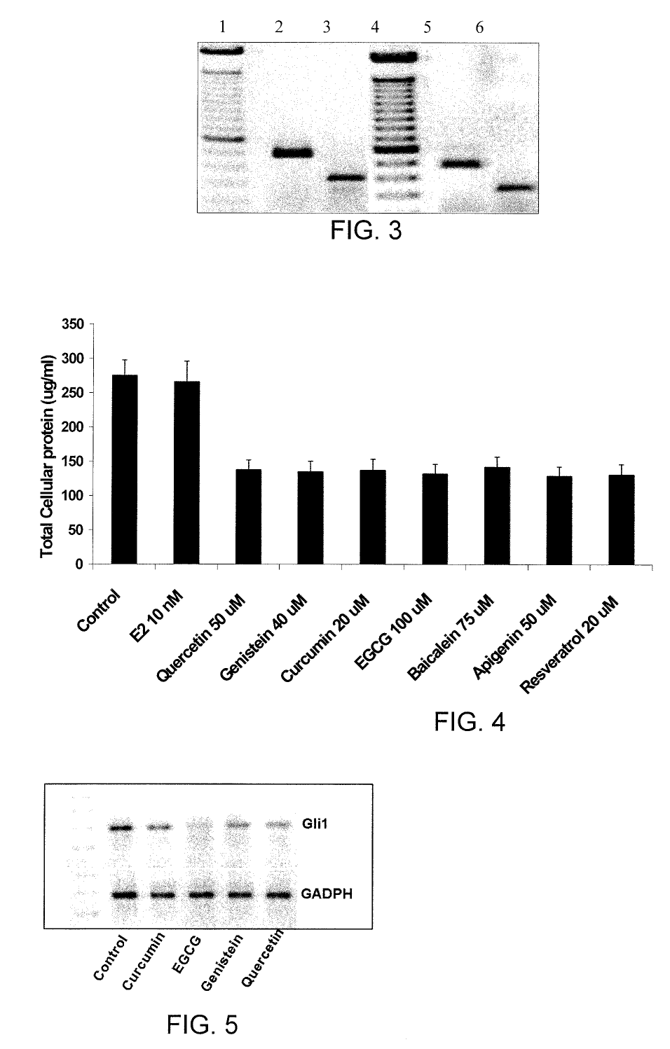

The expression profile of selected components of the Hedgehog-signaling pathway in the TRAMP-C2 cells was characterized. SHH, Gli1, Gli2 and Gli 3 were found to be expressed in these cells as determined by RT-PCR (FIG. 2). The expression of estrogen receptors in the TRAMP-C2 cells was analyzed using RT-PCR and western blots. Both receptors were found to be present in this cell line (FIG. 3). 100 bp ladder was loaded in lanes 1 and 4 of gels A & B, respectively. ERα primers span exons 5-8, ERβ primers span exons 8-9.

### Example 2

### Effects of Phytoestrogens on Shh Signaling in TRAMP-C2 and Shh Light II Cells

It has been shown that phytoestrogens in common herbs regulate prostate cancer cell growth in vitro (Shenouda et al., 2004). In order to determine whether these phytoestrogens also regulate cells derived from the mouse model of prostate cancer, the TRAMP-C2 cell line was treated with the soy phytoestrogen genistein and growth inhibition IC50 doses were determined (FIG. 4). Effects of the phytoestrogens genistein and EGCG on expression of these Hedgehog-pathway components were also determined by quantitative RT-PCR. Cyclopamine was used as a control. As shown in FIG. 5, genistein down-regulates Gli1 expression compared to the untreated control. GAPDH was used as an internal control.

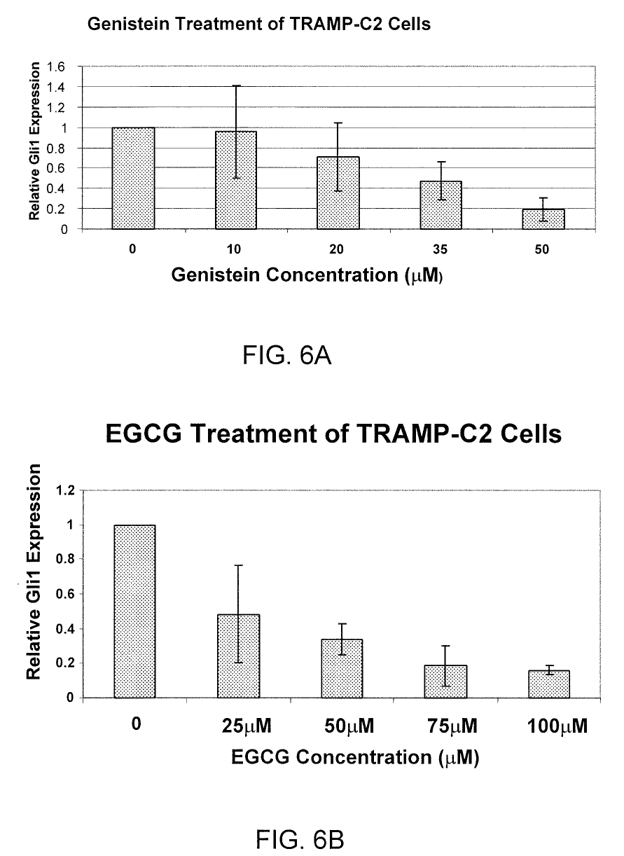

Another experiment was carried out to determine the dosage response of these phytoestrogen on Gli1 expression in the TRAMP-C2 cell line. Increasing amounts of genistein or EGCG were added to cell cultures of TRAMP-C2, and the levels of Gli1 mRNA was determined by real time RT-PCR. The results of this experiment verifies a correlation between phytoestrogen treatment and Hedgehog activity (FIG. 6). A decrease in Gli1 mRNA levels by more than 80% was observed at the highest treatment concentrations of genistein or EGCG. This observation suggests that phytoestrogens are likely to be major regulators, rather than merely effectors of the Hedgehog signaling pathway.

The RT-PCR results also showed that genistein and EGCG were both able to reduce Patched1 mRNA levels by 70%. Resveratrol also reduced Gli1 and Patched1 mRNAs by 60% and 50%, respectively. Approximate IC50 values for pathway repression were determined as 30 μM for genistein, 20 μM for EGCG, and 5 μM for resveratrol. These concentrations may be easily obtainable pharmacologically in humans. Another experiment showed that Gli1 protein concentration in LNCaP cells was also significantly reduced by all three compounds, genistein, EGCG, and resveratrol, as determined by Western blot analysis. Thus, Genistein, EGCG, and resveratrol may regulate prostate cancer via inhibition of the Hedgehog signaling pathway.

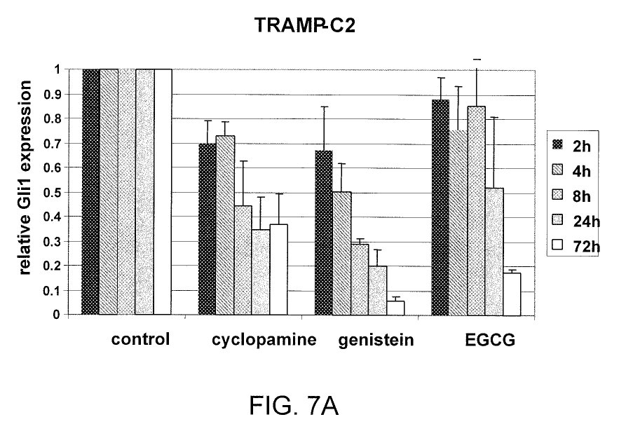

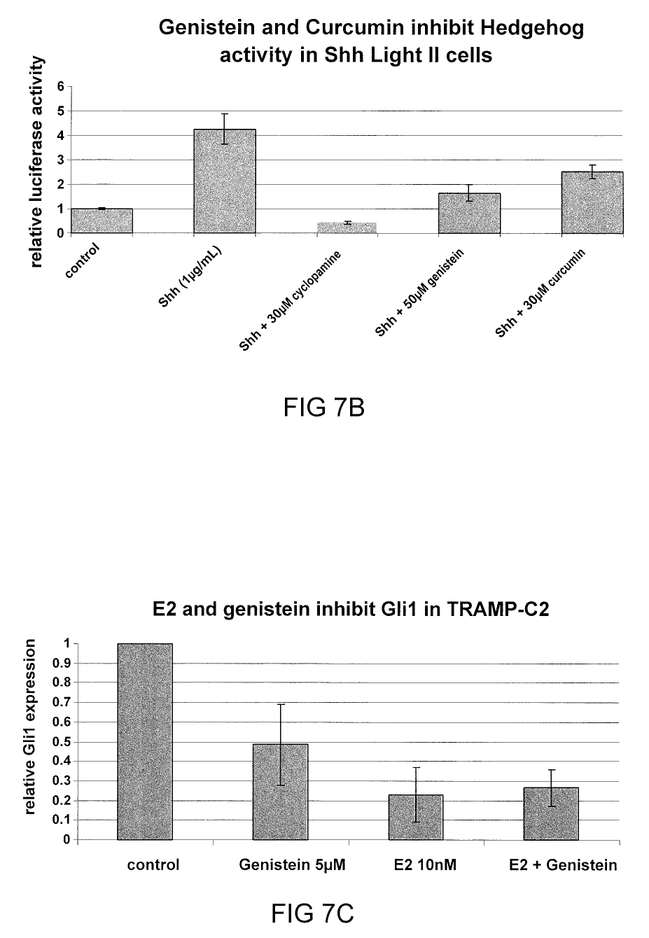

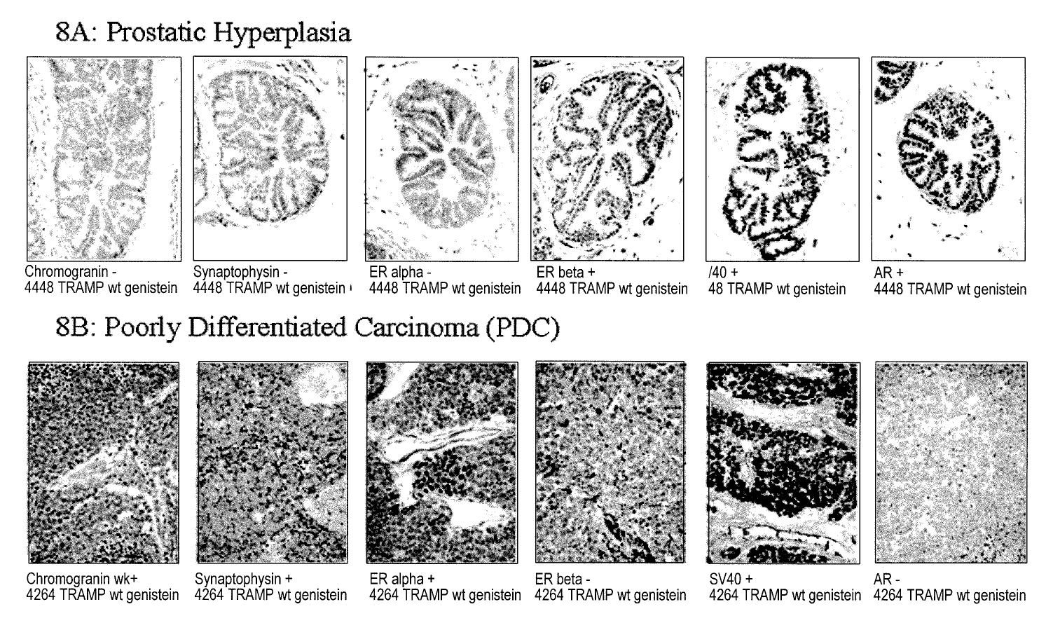

FIG. 7A shows result of a time course in which the concentration of Gli1 mRNA in the TRAMP-C2 cells are measured by real-time RT-PCR at multiple time points for 72 hrs following the addition of genistein, EGCG, or cyclopamine into the culture medium. The suppression of Gli1 mRNA concentration by genistein and EGCG as used at a final concentration of 50 μM and 100 μM, respectively, was more pronounced than the effect of cyclopamine at a final concentration of 30 μM.

The effects of various phytoestrogens on Hedgehog signaling in a non-tumor cell line Shh Light II were also assessed (FIG. 7B). Shh Light II cells were obtained from the ATCC. These cells, containing the Gli1 responsive promoter driving a luciferase reporter, are a standard tool used to assay Hedgehog pathway activity. Meloni et al., 2006. Cells were allowed to reach confluency in 24-well plates and subsequently treated with the selected phytoestrogens at the amount indicated on the figure, along with the stimulatory sonic hedgehog protein (obtained from R&D Systems). The cells were then grown for 24 hours in phenol red-free DMEM supplemented with 0.5% charcoal stripped fetal bovine serum. Cells were then lysed, and the luciferase and renilla activities of the cell lysates were measured. As shown in FIG. 7B, similar to the effects exerted by cyclopamine, both genistein and curcumin also inhibit hedgehog activity as evidenced by the decrease in luciferase reporter activity.

In order to test whether the inhibitory effect of genistein on Gli1 transcription is through ERα or ERβ, experiments were conducted to determine whether 17β-estradiol (E2) works in the same manner as genistein (FIG. 7C). Strong inhibition of Gli1 expression was observed with 10 nM E2. Addition of genistein did not enhance this inhibition by E2, suggesting that both compounds may act through the Estrogen Receptors.

### Example 3

### Anti-Tumor Effects of Phytoestrogens as Dietary Supplements in TRAMP Mice

The influence of estrogen on prostate cancer has been previously described in the Background section. In order to determine the effect of genistein on prostate tumor development in the TRAMP model, in the first experiment, groups of ˜30 male mice, either ERαKO/TRAMP, ERβKO/TRAMP or ERWT/TRAMP were fed diets containing 0 or 300 mg genistein/kg (See Table 1). Mice were fed the diets from weaning and were euthanized at 5 months of age. Prostates were dissected and prepared for histopathology. The tumor stage was assessed using ratings of normal (N), hyperplasia (HYP), prostatic intraepithelial neoplasia (PIN), well differentiated carcinoma (WDC), moderately differentiated carcinoma (MDC) or poorly differentiated carcinoma (PDC) See Wechter et al., 2000; also see http://thegreenberglab.fhcrc.org. To summarize the data, non-cancer was defined as N, HYP or PIN and cancer was defined as WDC, MDC or PDC (Table 1) The prostate tumor incidence was compared between mice fed with genistein diet and those fed with casein. A significant protective effect of genistein was observed in the ERWT/TRAMP mice but not in the ERαKO/TRAMP and ERβKO/TRAMP mice, suggesting that both ERα and ERβ are essential for the response to genistein (See Table 2).

TABLE 1
Incidence of prostate tumorigenesis in ERαKO/TRAMP, ERβKO/TRAMP
and ERWT/TRAMP mice fed with 300 mg genistein/kg diet

| | | | Phenotype | | | | | |
|---|---|---|---|---|---|---|---|---|
| Genotype | | | Non-Cancer | | | Cancer | | |
| All | | | Normal | HYP | PIN | WDC | MDC | PDC |
| TRAMP | Diet | n | (Stage1) | (2) | (3) | (4) | (5) | (6) |
| ERWT | Casein | 74 | 1(1.4%) | 12(16.2%) | 11(14.9%) | 35(47.2%) | 0 | 15(20.3%) |
| ERWT | Genistein | 54 | 0 | 15(28%) | 18(33%) | 7(13%) | 1(2%) | 13(24%) |
| ERαKO | Casein | 79 | 0 | 2(3.0%) | 4(5%) | 68(86%) | 1(1%) | 4(5%) |
| ERαKO | Genistein | 26 | 1(3.8%) | 0 | 1(3.8%) | 23(88.6%) | 1(3.8%) | 0 |
| ERβKO | Casein | 21 | 0 | 0 | 5(24%) | 6(28%) | 0 | 10(48%) |
| ERβKO | Genistein | 21 | 0 | 0 | 4(19%) | 7(33%) | 0 | 10(48%) |

TABLE 2
Statistical Comparisons
1. CANCER (4-6) versus NON CANCER (1-3)

| | | |
|---|---|---|
| a- ER-WT casein versus ER-WT Genistein | P = 0.0013 | Significant |
| b- ERαKO casein versus ERαKO genistein | P = 0.68 | Non |
| | | significant |
| c- ERβKO casein versus ERβKO genistein | P = 0.71 | Non |
| | | Significant |

2. PDC versus NON-PDC

| | | |
|---|---|---|
| a- ER-WT casein versus ERαKO casein | P = 0.0002 | Significant |
| b- ER-WT casein versus ERβKO casein | P = 0.019 | Significant |
| c- ER-WT genistein versus ERαKO genistein | P = <0.0001 | Significant |
| d- ER-WT genistein versus ERβKO genistein | P = 0.047 | Significant |

In order to assess the effects of other phytoestrogens on prostate cancer, TRAMP mice with wild-type ER were fed diets supplemented with various combinations of Curcumin (from Dr. Acharan, and added to a final concentration of 10 gram/kg of the diet), Resveratrol (from Sigma, and added to a final concentration of 10 mg/kg of the diet), EGCG (from Sigma, and added to a final concentration of 100 mg/kg of the diet), Genistein (from LC laboratories, and added to a final concentration of 250 mg/kg of the diet), Quercetin (from Sigma, and added to a final concentration of 1 gram/kg of the diet), Baicalein (from Dr. Acharan, and added to a final concentration of 150 mg/kg of the diet), and Apigenin (from LC laboratories, and added to a final concentration of 15 mg/kg of the diet). Curcumin is also commercially available from NOW Foods (Bloomingdale Ill.), and Baicalein is also commercially available from Caymen Chemicals (Ann Arbor, Mich.). The results of this study indicate that both Mix 3 and Mix 4 significantly delay and/or prevent the occurrence of prostate cancer in these mice (Table 3).

TABLE 3
Incidence of prostate tumorigenesis in ERWT/TRAMP mice fed
with diet supplemented with combination of phytoestrogens

| | | | Phenotype | | | | | |
|---|---|---|---|---|---|---|---|---|
| Genotype | | | Non-Cancer | | | Cancer | | |
| all | | | Normal | HYP | PIN | WDC | MDC | PDC |
| TRAMP | Diet | N | 1 | 2 | 3 | 4 | 5 | 6 |
| ERWT | Casein | 22 | 0 | 4.5% | 9% | 63.6% | 0 | 22.9% |
| ERWT | Mix 3 | 20 | 0 | 10% | 40% | 40% | 0 | 10% |
| ERWT | Mix 4 | 18 | 0 | 44.5% | 22.2% | 11.1% | 0 | 22.2% |
| ERWT | Pure 7 | 19 | 0 | 0 | 57.8% | 26.3% | 0 | 15.9% |

Mix 3 = Curcumin + Resveratrol + EGCG
Mix 4 = Genistein + Quercetin + Baicalein + Apigenin
Pure 7 = Mix 3 + Mix 4

### Example 4

### Immunohistochemical Analyses of the TRAMP Mouse

Immunohistochemical analyses of neuroendocrine marker and hormone receptor expression in the prostates from TRAMP mice were conducted. Shown in FIG. 8 are representative tissue samples from two ERWT/TRAMP mice fed genistein with HYP (FIG. 8A) from the dorsal lobe or PDC (FIG. 8B) from the ventral lobe. In both panels, the brown regions represent immunoreactivity. HYP prostate epithelium does not express chromogranin and synaptophysin (cytoplasmic neuroendocrine (NE) markers) or ERα (nuclear receptor). There is expression of ERβ and androgen receptor in epithelial and stromal cells, as has been reported for normal prostate epithelium in man and mouse (Prins et al, 1998). The SV40 immunoreactivity confirms the expression of the transgene in the nucleus of the prostate epithelium. The immunoreactivity to this panel of antibodies of the HYP prostate was not different from those fed casein. As shown in FIG. 8B, these markers differed in PDC tissues. There was no difference between casein and genistein fed mice. In contrast to the HYP prostate, NE carcinoma in this TRAMP model did express neuroendocrine markers, with 80% of sections expressing synaptophysin, and 30% expressing chromogranin. Androgen receptor reactivity diminished from occasional weak positives to uniformly negative. Transgene expression was maintained in NE cells.

The pattern of estrogen receptor immunoreactivity switched, with nearly complete loss of ERβ expression and gain of ERα expression. The pattern of ERα immunoreactivity was predominantly in clusters of NE cells scattered throughout the tumor mass and not in all NE cells. In data not shown, ERα expression was also observed in PIN and well-differentiated adenocarcinoma cells in both diet groups. Hence, distinct cancer origins appear to occur in the TRAMP prostate and these are independent of diet.

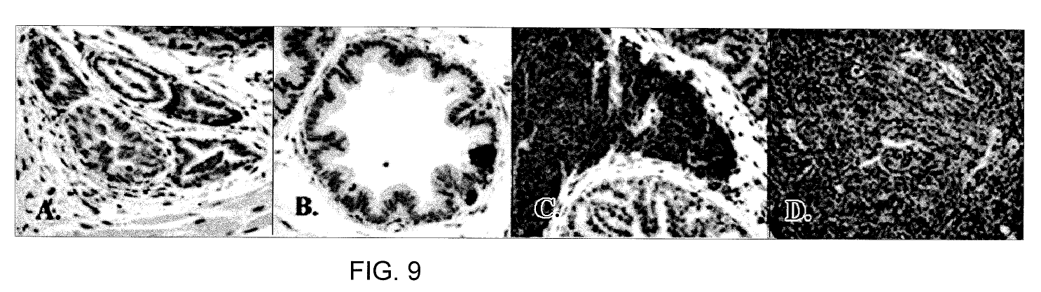

In the TRAMP studies described above, PDC appeared to arise predominately in the ventral lobe of the prostate, as also reported by Kaplan-Lefko and Greenberg, 2003. The PDC observed was characterized as being heterogeneous for positive staining with synaptophysin. The PDC likely started as a focus of neuro-endocrine cell proliferation, then progressively penetrated the tubules, and proliferated in the stroma, encroaching on adjacent tubules and eventually surrounding the urethra (See progression in FIG. 9A to 9D). FIG. 9A shows basal cells in the prostate ducts are positive for synaptophysin, a marker of neuroendocrine cells. The prostate secretory epithelium is synaptophysin negative. FIG. 9B shows a focus of neuroendocrine cell proliferation, the earliest stage of neuroendocrine-like neoplasia. FIG. 9C shows the neoplastic neuroendocrine cells penetrate through the wall of the prostate tubule and proliferate in the stroma, encroaching adjacent tubules. FIG. 9D shows neoplastic neuroendocrine cells eventually surround the urethra.

The incidence of PDC has been reported to increase with androgen ablation. In the TRAMP studies, the incidence of well differentiated carcinoma (WDC) was predominantly found in the dorsal lateral prostate and in the ERαKO casein group was significantly (P<0.00001) higher compared to ERWT casein. On the other hand, the ERαKO casein group has a significantly (p<0.0006) lower frequency of PDC compared to ERWT casein (Table 1 above). However, the incidence of PDC was higher in ERβKO than in ERWT casein (Table 1). One possible explanation is that ERα and ERβ may have opposing roles in the prostate in regard to prostate cancer and that ERα facilitates the development of PDC, while ERβ plays a protective role in prostate cancer. This role is supported in the literature. See e.g., Imamov et al., 2004, and Signoretti et al, 2001. The balance between ERα and ERβ seems to have a significant role in the development of PDC. These data, in conjunction with soy genistein's preferential binding to ERβ, suggest a mechanism by which phytoestrogens may help prevent and/or suppress prostate cancer by binding and regulating ERα, ERβ or both.

While the foregoing instrumentalities have been described in some detail for purposes of clarity and understanding, it will be clear to one skilled in the art from a reading of this disclosure that various changes in form and detail can be made without departing from the true scope of the invention. For example, all the techniques and apparatus described above may be used in various combinations. All publications, patents, patent applications, or other documents cited in this application are incorporated by reference in their entirety for all purposes to the same extent as if each individual publication, patent, patent application, or other document were individually indicated to be incorporated by reference for all purposes.

### REFERENCES

Additional information is found in the following publications and references cited within this disclosure, which are hereby incorporated by reference.
Barnes, S., Peterson, G., Grubbs, C., and Setchell, K. D. Potential role of dietary isoflavones in the prevention of cancer. Advances in Experimental Medicine and Biology, 354: 135-147, 1994.
Datta, S. and M. W. Datta, Sonic Hedgehog signaling in advanced prostate cancer. Cell. Mol. Life Sci. 63 (2006) 435-448.
Foster, B. A., Gingrich, J. R., Kwon, E. D., Madias, C., and Greenberg, N. M. Characterization of prostatic epithelial cell lines derived from transgenic adenocarcinoma of the mouse prostate (TRAMP) model. Cancer Res, 57: 3325-3330, 1997.
Goetzl M A, Vanveldhuizen P J, Thrasher J B., Effects of soy phytoestrogens on the prostate. Prostate Cancer Prostatic Dis. 2007, Epub ahead of print.
Greenberg, N. M., DeMayo, F., Finegold, M. J., Medina, D., Tilley, W. D., Aspinall, J. O., Cunha, G. R., Donjacour, A. A., Matusik, R. J., and Rosen, J. M. Prostate cancer in a transgenic mouse. Proceedings of the National Academy of Sciences of the United States of America, 92: 3439-3443, 1995.
Guerini, V., Sau, D., Scaccianoce, E., Rusmini, P., Ciana, P., Maggi, A., Martini, P. G., Katzenellenbogen, B. S., Martini, L., Motta, M., and Poletti, A. The androgen derivative 5alpha-androstane-3beta, 17beta-diol inhibits prostate cancer cell migration through activation of the estrogen receptor beta subtype. Cancer Res, 65: 5445-5453, 2005.
Imamov, O., Morani, A., Shim, G. J., Omoto, Y., Thulin-Andersson, C., Warner, M., and Gustafsson, J. A. Estrogen receptor beta regulates epithelial cellular differentiation in the mouse ventral prostate. Proc Natl Acad Sci U S A, 101: 9375-9380, 2004.
Jemal A, Siegel R, Ward E, Murray T, Xu J, Thun M J. (2007). Cancer Statistics, 2007. CA Cancer J Clin 57(1):43-66.
Kaplan-Lefko, P. J., Chen, T. M., Ittmann, M. M., Barrios, R. J., Ayala, G. E., Huss, W. J., Maddison, L. A., Foster, B. A., and Greenberg, N. M. Pathobiology of autochthonous prostate cancer in a pre-clinical transgenic mouse model. Prostate, 55: 219-237, 2003.

Karhadkar, S. S., Bova, G. S., Abdallah, N., Dhara, S., Gardner, D., Maitra, A., Isaacs, J. T., Berman, D. M., and Beachy, P. A. Hedgehog signalling in prostate regeneration, neoplasia and metastasis. Nature, 431: 707-712, 2004.

Keeler R F. Teratogenic compounds of Veratrum californicum (Durand) X. Cyclopia in rabbits produced by cyclopamine. Teratology 1970; 3(2):175-180.

Kuiper G G, Carlsson B, Grandien K, Enmark E, Häggblad J, Nilsson S, Gustafsson J A. Comparison of the ligand binding specificity and transcript tissue distribution of estrogen receptors alpha and beta. Endocrinology. 1997 138(3):863-70
Meloni A R, Fralish G B, Kelly P, Salahpour A, Chen J K, Wechsler-Reya R J, Lefkowitz R J, Caron M G. Smoothened signal transduction is promoted by G protein-coupled receptor kinase 2. Mol Cell Biol 2006; 26(20):7550-7560.
Mentor-Marcel, R., Lamartiniere, C. A., Eltoum, I. E., Greenberg, N. M., and Elgavish, A. Genistein in the diet reduces the incidence of poorly differentiated prostatic adenocarcinoma in transgenic mice (TRAMP). Cancer Research, 61: 6777-6782, 2001.
Prins, G. S., Marmer, M., Woodham, C., Chang, W., Kuiper, G., Gustafsson, J.-A., and Birch, L. Estrogen receptor-β messenger ribonucleic acid ontogeny in the prostate of normal and neonatally estrogenized rats. Endocrinology, 139: 874-883, 1998.
Ruiz i Altaba, A., Sanchez, P., and Dahmane, N. Gli and Hedgehog in cancer: tumours, embryos and stem cells. Nat Rev Cancer, 2: 361-372, 2002.
Sanchez P, Hernandez A M, Stecca B, Kahler A J, DeGueme A M, Barrett A, Beyna M, Datta M W, Datta S, Ruiz i Altaba A. Inhibition of prostate cancer proliferation by interference with SONIC HEDGEHOG-GLI1 signaling. Proc Natl Acad Sci USA 2004; 101(34):12561-12566.
Sanchez, P., Clement, V., and Ruiz i Altaba, A. Therapeutic targeting of the Hedgehog-GLI pathway in prostate cancer. Cancer Res, 65: 2990-2992, 2005.
Shenouda, N., Zhou, C., Browning, J., Ansell, P., Sakla, M., and Lubahn, D. Phytoestrogens in commom herbs regulate prostate cancer cell growth in vitro. Nutrition and Cancer, 49: 201-209, 2004.
Signoretti, S. and Loda, M. Estrogen receptor beta in prostate cancer: brake pedal or accelerator? Am J Pathol, 159: 13-16, 2001.
Takimoto C H, Glover K, Huang X, Hayes S A, Gallot L, Quinn M, Jovanovic B D, Shapiro A, Hernandez L, Goetz A, Llorens V, Lieberman R, Crowell J A, Poisson B A, Bergan R C. Phase I pharmacokinetic and pharmacodynamic analysis of unconjugated soy isoflavones administered to individuals with cancer. Cancer Epidemiol Biomarkers Prev. 2003 November; 12(11 Pt 1):1213-21.
Wechter, W. J., Leipold, D. D., Murray, E. D., Quiggle, D., McCracken, J. D., Barrios, R. S., and Greenberg, N. M. E-7869 (R-flurbiprofen) inhibits progression of prostate cancer in the TRAMP mouse. Cancer Res., 60. 2203-2208, 2000.

We claim:

1. A composition comprising an effective amount of an estrogenic agent, wherein said estrogenic agent in said effective amount is capable of inhibiting the Hedgehog signaling pathway in a cell.

2. The composition of claim 1, wherein said estrogenic agent comprises a phytoestrogen, or its analog, derivative, ester, pharmaceutically acceptable salt, hydrate, or any combination thereof.

3. The composition of claim 2, wherein the phytoestrogen is at least one member selected from the group consisting of Genistein, Quercetin, Baicalein, Apigenin, Daidzein, Neoxanthin, Spinacetin, Patuletin, Luteolin, Curcumin, Resveratrol, EGCG and derivatives thereof.

4. The composition of claim 1, wherein said estrogenic agent comprises two or more phytoestrogens selected from the group consisting of Genistein, Quercetin, Baicalein, Apigenin, Daidzein, Neoxanthin, Spinacetin, Patuletin, Luteolin, Curcumin, Resveratrol, EGCG and derivatives thereof.

5. The composition of claim 1, wherein said effective amount is an amount of the estrogenic agent that is effective in reducing the expression of at least one Gli gene by at least 40%.

6. The composition of claim 5, wherein the at least one Gli gene is selected from the group consisting of Gli1, Gli2 and Gli3.

7. The composition of claim 1 wherein said estrogenic agent in said effective amount is capable of inhibiting the Hedgehog signaling pathway in a cell by binding to an estrogen receptor in said cell.

8. A method for treating cancer, or for preventing the incidence or the recurrence of cancer in an individual, comprising administering to said individual an effective amount of an estrogenic agent, wherein said estrogenic agent in said effective amount is capable of inhibiting the Hedgehog signaling pathway in certain cells of said individual.

9. The method of claim 8, wherein said estrogenic agent in said effective amount is capable of inhibiting the Hedgehog signaling pathway in said individual by binding to an estrogen receptor in said individual.

10. The method of claim 8, wherein said estrogenic agent comprises a phytoestrogen, or its analog, derivative, ester, pharmaceutically acceptable salt, hydrate, or any combination thereof.

11. The method of claim 10, wherein the phytoestrogen is at least one member selected from the group consisting of Genistein, Quercetin, Baicalein, Apigenin, Daidzein, Neoxanthin, Spinacetin, Patuletin, Luteolin, Curcumin, Resveratrol, EGCG and derivatives thereof.

12. The method of claim 8, wherein said estrogenic agent comprises two or more phytoestrogens selected from the group consisting of Genistein, Quercetin, Baicalein, Apigenin, Daidzein, Neoxanthin, Spinacetin, Patuletin, Luteolin, Curcumin, Resveratrol, EGCG and derivatives thereof.

13. The method of claim 8, wherein said effective amount is an amount of the estrogenic agent that is effective in reducing the expression of at least one Gli gene by at least 40%.

14. The method of claim 13, wherein the at least one Gli gene is selected from the group consisting of Gli1, Gli2 and Gli3.

15. A method for treating cancer, or for preventing the incidence or the recurrence of cancer in an individual, comprising administering to said individual an effective amount of an agent, wherein said agent in said effective amount is capable of binding at least one estrogen receptor in the individual, thereby inhibiting the Hedgehog signaling pathway in said individual.

16. The method of claim 15, wherein said agent is an estrogenic agent.

17. A method for inhibiting Hedgehog signaling pathway in an individual with aberrant Hedgehog signaling, comprising:
a) determining whether Hedgehog signaling is abnormal in said individual; and
b) administering to said individual a Hedgehog signaling inhibiting effective amount of an estrogenic agent.

18. The method of claim 17, wherein said estrogenic agent comprises a phytoestrogen, or its analog, derivative, ester, pharmaceutically acceptable salt, hydrate, or any combination thereof.

19. The method of claim 18, wherein the phytoestrogen is at least one member selected from the group consisting of Genistein, Quercetin, Baicalein, Apigenin, Daidzein, Neoxanthin, Spinacetin, Patuletin, Luteolin, Curcumin, Resveratrol, EGCG and derivatives thereof.

20. The method of claim 17, wherein said estrogenic agent comprises two or more phytoestrogens selected from the group consisting of Genistein, Quercetin, Baicalein, Apigenin, Daidzein, Neoxanthin, Spinacetin, Patuletin, Luteolin, Curcumin, Resveratrol, EGCG and derivatives thereof.

21. A method for inhibiting Hedgehog signaling pathway in a cell, comprising contacting said cell with an estrogenic agent, said estrogenic agent being present in an amount that is effective in inhibiting expression of at least one Gli gene by at least 40%.

22. The method of claim 21, wherein the Gli gene is selected from the group consisting of Gli1, Gli2 and Gli3.

23. A method of screening for a potential therapeutic agent for treating or preventing cancer, said method comprising the steps of:
(a) contacting a cell with an intact Hedgehog signaling pathway with a candidate molecule;
(b) monitoring changes in the activation of the Hedgehog signaling pathway;
(c) determining whether said molecule is capable of inhibiting the Hedgehog signaling pathway; and
(d) identifying said molecule as a potential therapeutic agent if it is determined to be capable of inhibiting the Hedgehog signaling pathway.

24. A molecule identified according to the method of claim 23 to be a potential therapeutic agent.

25. A method for treating cancer, or for preventing the incidence or the recurrence of cancer in an individual, comprising administering to said individual an effective amount of an estrogenic agent, wherein said estrogenic agent in said effective amount is capable of inhibiting the Hedgehog signaling pathway in certain cells of said individual, and said estrogenic agent comprises two or more phytoestrogens selected from the group consisting of Genistein, Quercetin, Baicalein, Apigenin, Daidzein, Neoxanthin, Spinacetin, Patuletin, Luteolin, Curcumin, Resveratrol, EGCG and derivatives thereof.

26. The method of claim 25, wherein said estrogenic agent is a mixture comprising Curcumin, Resveratrol and EGCG.

27. The method of claim 25, wherein said estrogenic agent is a mixture comprising Genistein, Quercetin, Baicalein, and Apigenin.
